(19) United States
(12) Patent Application Publication
(10) Pub. No.: US 2025/0093491 A1
(43) Pub. Date: Mar. 20, 2025

(54) Microwave Architecture for Passive Sensing Applications

(71) Applicant: Texas Tech University System, Lubbock, TX (US)

(72) Inventors: Changzhi Li, Lubbock, TX (US); Davi Valerio de Queiroz Rodrigues, El Paso, TX (US); Dongyang Tang, San Diego, CA (US)

(21) Appl. No.: 18/728,142

(22) PCT Filed: Jan. 13, 2023
(86) PCT No.: PCT/US2023/060650

§ 371 Date: Jul. 11, 2024

Related U.S. Application Data
(60) Provisional application No. 63/299,942, filed on Jan. 15, 2022.

(51) Int. Cl. G01S 13/62; A61B 5/0507

(52) U.S. Cl. CPC G01S 13/62; A61B 5/0507

(57) ABSTRACT

A microwave passive sensor includes a microwave receiver, a low noise amplifier coupled to the microwave receiver, a mixer coupled to the low noise amplifier, and a baseband amplifier coupled to the mixer.

### CROSS-REFERENCE TO RELATED APPLICATIONS

This application claims priority to U.S. Provisional Application Ser. No. 63/299,942, filed Jan. 15, 2022, the entire contents of which are incorporated herein by reference.

### STATEMENT OF FEDERALLY FUNDED RESEARCH

This invention was made with government support under Grant Nos. ECCS-1808613 and ECCS-2030094 awarded by the National Science Foundation. The government has certain rights in the invention.

### TECHNICAL FIELD OF THE INVENTION

The present invention relates in general to the field of microwave devices, and more particularly, to a microwave architecture for passive sensing applications.

### BACKGROUND OF THE INVENTION

Without limiting the scope of the invention, its background is described in connection with microwave devices.

Microwave passive sensing makes use of electromagnetic waves emitted by a third-party transmitter to detect and localize targets [1]. The changes on the wireless signals are decoded by monitoring the strength of the received signals, by comparing the propagation characteristics of the communication channel over time, or by correlating the signals arriving after being reflected at a moving target and a reference signal, which is the time-delayed version of the transmitted signal [2]-[8], [14]. By comparing the direct signal from the transmitter and the reflected signal from the target of interest, the motion of the target can be detected. With the rapid growth of the Internet of Things (IoT) and satellite internet, wireless signals are ubiquitously present in the ambient air (e.g., Wi-Fi access points, Bluetooth signals, wireless power transfer, electromagnetic waves leaked from commercial microwave ovens, etc.).

In addition, the fast-growing wireless communication technologies require more and more radio spectrum and have started to affect conventional radars because of the potential interference. For example, major U.S. airlines recently warned that the new C-Band 5G service could potentially interfere with the altimeter and cause a significant number of aircraft to be unusable. The automotive and consumer electronics industries have also been looking for interference mitigation approaches due to the increasing number of cars and appliances that rely on wireless devices operating at the same frequency for communication or sensing purposes. Therefore, microwave passive sensing technology has tremendous opportunities, and it has gained attention in recent decades.

Many efforts have been made to evaluate the feasibility of microwave passive sensing [2]-[13]. The prior arts have demonstrated the possibility of indoor positioning [2], human targets detection [3], [10], human activity and gesture recognition [4], [5], [8], [9], and vital sign monitoring [6], [11] using passive sensing technologies in the Wi-Fi frequency band. The existing microwave passive sensing works can be classified into two main categories based on whether additional RF receivers are needed or not.

One of the categories extracts target information from existing wireless communication devices, such as Wi-Fi access points [2], [5], [6]. One of the advantages of this category is that the transceivers in the Wi-Fi access points are synchronized, and it has the information for both the transmitted signal and the target reflected signal. However, because the Wi-Fi access points are not designed to detect targets, they do not directly provide targets' information as conventional radars do. In those works, the target detection needs to be extracted from the available data in the Wi-Fi system, such as received signal strength (RSS) and channel state information (CSI). For example, Wi-Fi-based RSS has been used for indoor occupancy sensing [3]. However, it suffers from unpredictable fluctuations in the communication link due to the existence of multiple reflections and scattering paths even in a static illumination scene. CSI-based systems extract fine-grained measurements obtained after the interaction between wireless signals and surrounding moving objects/human subjects. For example, [4] employs CSI data to provide remote finger gesture recognition. In [5], CSI-based human activity recognition is studied. The major challenge for CSI systems is the constant changes in the surrounding environment, which considerably affects the communication channel requiring a training phase for each environment. Some of the disadvantages of this category is that the systems require special Wi-Fi network interface cards to access the necessary data from the Wi-Fi access points and specially designed computationally intensive algorithms. While it is attractive to achieve the target detection without additional RF devices, the existing hardware and software limit the flexibility and performance of the system.

The other category of microwave passive sensing requires customized RF receivers but does not require access to any information inside the Wi-Fi access point [8]-[13]. Since the theories and algorithms used to extract the target for this category are identical to those used in conventional radars, they are called passive Wi-Fi radars (PWR). The mixer in a conventional radar needs two inputs: a local oscillator (LO) and an RF input. The LO signal is used as the reference signal to down-convert the RF input. The challenge for the PWR is generating the reference signal or the LO signal for the mixer since it does not have a hardware connection to the transmitter. PWR systems allow the detection of moving targets through the cross-ambiguity function (CAF), which is evaluated by comparing the reference and the surveillance channels without any prior modification to existing Wi-Fi devices [7]-[8]. Two separated channels were used to solve this problem in [8]-[10]: a reference channel and a surveillance channel. It was assumed that the reference channel contained the signal from the transmitter while the surveillance channel included the reflected signal from the target. PWR with one channel was proved to be feasible with an injection-locking oscillator [11]. Notably, PWR-based approaches are heavily affected by direct signal interference (DSI) and require DSI removal algorithms to obtain accurate Doppler information.

Now referring to FIG. 1, a block diagram of a conventional Doppler radar 100 [15] is shown in accordance with the prior art. In a conventional Doppler radar [29], and [30]: an oscillator 102 generates the RF signal; the oscillator output is split by a power divider 102 into two signals, one of them T(t) is amplified by a power amplifier 106 and transmitted towards the target 108 using the transmitter antenna 110, the other one is sent to the LO port 112 of the mixer 114; an LNA 116 amplifies the phase-modulated signal R(t) reflected by the moving target 108 in the receiver antenna 118; the amplified signal is sent to the RF input port 120 of the mixer 114; and the mixer 114 down-converts the RF input with the reference signal (LO) to the baseband signals I(t) and Q(t), which carry the target's motion information.

Although the existing passive sensing techniques may accomplish the goal of taking advantage of pervasive Wi-Fi signals, they still require the use of customized RF transceivers and algorithms of relatively high computational cost to achieve robust detection.

According there is a need for a new microwave architecture for passive sensing applications.

### SUMMARY OF THE INVENTION

Microwave architectures for passive sensing applications are disclosed herein. The capability to simultaneously retrieve both the transmitted signal from a non-cooperative microwave source and the signals scattered by a target is the key to enable the identification of Doppler frequencies associated with the target of interest. Neither hardware modification nor synchronization to the signal source is needed, and a simple Fourier-based analysis of the baseband responses is used to extract the Doppler information of a moving target. As a result, the radio-frequency topology leverages current and next generation Wi-Fi, Bluetooth, and wireless power transfer infrastructure to make best use of RF radiations, spectrum, and wireless networks for ubiquitous smart home, health care, and smart living by passively using ambient RF signals for the identification of a target's motion.

Moreover, the passive sensing applications disclosed herein enable the possibility of microwave passive sensing at most places on our planet. Since microwave passive sensing does not transmit its own radio frequency (RF) signals, it inherently reduces cost and power compared with conventional radar sensors. Moreover, it provides a solution to address the interference with wireless communication signals and radar signals, and contribute to improve the spectrum efficiency.

One embodiment of the present disclosure provides a microwave passive sensor that includes a microwave receiver, a low noise amplifier coupled to the microwave receiver, a mixer coupled to the low noise amplifier, and a baseband amplifier coupled to the mixer.

In one aspect, the low noise amplifier comprises two cascaded low noise amplifiers. In another aspect, the mixer is radio frequency (RF) coupled to the low noise amplifier with a first capacitor, and the baseband amplifier is RF coupled to the mixer with a second capacitor. In another aspect, the mixer comprises a diode coupled to ground. In another aspect, the mixer comprises a passive quadrature mixer. In another aspect, the mixer comprises: a first resistor coupled between a voltage source and a mixer output; an inductor coupled between the mixer output and a mixer input; and a diode coupled between the mixer input and a ground. In another aspect, the baseband amplifier comprises: a second resistor coupled between a voltage source and a negative input of an amplifier; a third resistor and a fourth capacitor connected in parallel between the negative input of the amplifier and a ground; a fourth resistor coupled between a mixer output and a positive input of the amplifier; and a fifth resistor and a third capacitor connected in parallel between the positive input of the amplifier and an output of the amplifier. In another aspect, a gain block amplifier is coupled to the low noise amplifier, and a power divider is coupled between the gain block amplifier and the mixer. In another aspect, the power divider separates a signal received by the microwave receiver into a LO signal and a RF signal. In another aspect, an analog to digital converter is coupled to the baseband amplifier, and a processor or computer coupled to the analog to digital converter. In another aspect, the microwave passive sensor is not synchronized or cooperative with a microwave source. In another aspect, the microwave source comprises a Wi-Fi access point or a Bluetooth signal source. In another aspect, the microwave passive sensor provides Doppler information of a moving target based on reflected signals from the moving target and direct-path signals from the microwave source. In another aspect, the moving target comprises a human, an animal, an object, a fluid, a human activity, a human gesture, a human vital sign or an animal vital sign. In another aspect, the microwave passive sensor is tunable to different frequencies or scans a range of frequencies.

Another embodiment of the present disclosure provides a method for passively detecting a movement of a target. The method includes receiving signals using a microwave receiver, amplifying the received signals using a low noise amplifier coupled to the microwave receiver, separating the amplified received signals into a reflected signal from the target and a direct signal from a microwave source, producing I/Q baseband signals from the reflected signal and the direct signal using a mixer, amplifying the I/Q baseband signals using a baseband amplifier, and detecting the movement of the target using the amplified I/Q baseband signals.

In one aspect, the low noise amplifier comprises two cascaded low noise amplifiers. In another aspect, the mixer is radio frequency (RF) coupled to the low noise amplifier with a first capacitor, and the baseband amplifier is RF coupled to the mixer with a second capacitor. In another aspect, the mixer comprises a diode coupled to ground. In another aspect, the mixer comprises a passive quadrature mixer. In another aspect, the mixer comprises: a first resistor coupled between a voltage source and a mixer output; an inductor coupled between the mixer output and a mixer input; and a diode coupled between the mixer input and a ground. In another aspect, the baseband amplifier comprises: a second resistor coupled between a voltage source and a negative input of an amplifier; a third resistor and a fourth capacitor connected in parallel between the negative input of the amplifier and a ground; a fourth resistor coupled between a mixer output and a positive input of the amplifier; and a fifth resistor and a third capacitor connected in parallel between the positive input of the amplifier and an output of the amplifier. In another aspect, the amplified received signals are further amplified by a gain block amplifier coupled to the low noise amplifier, and the amplified received signals are separated by a power divider coupled between the gain block amplifier and the mixer. In another aspect, the movement of the target is detected by performing a Fourier-based analysis of the amplified I/Q baseband signals using a processor or computer. In another aspect, the method further comprises providing an analog to digital converter coupled to the baseband amplifier, and providing a processor or computer is coupled to the analog to digital converter. In another aspect, the microwave receiver is not synchronized or cooperative with the microwave source. In another aspect, the microwave source comprises a Wi-Fi access point or a Bluetooth signal source. In another aspect, the method further comprises providing Doppler information of a moving target based on reflected signals from the moving target and direct-path signals from a microwave source. In another aspect, the moving target comprises a human, an animal, an object, a fluid, a human activity, a human gesture, a human vital sign or an animal vital sign. In another aspect, further comprising tuning the microwave passive sensor to different frequencies or scanning a range of frequencies.

Another embodiment of the present disclosure provides a system that includes a microwave passive sensor, a microwave source, and a processor or computer coupled to the microwave sensor. The microwave passive sensor comprises a microwave receiver, a low noise amplifier coupled to the microwave receiver, a mixer coupled to the low noise amplifier, and a baseband amplifier coupled to the mixer.

In one aspect, the low noise amplifier comprises two cascaded low noise amplifiers. In another aspect, the mixer is radio frequency (RF) coupled to the low noise amplifier with a first capacitor, and the baseband amplifier is RF coupled to the mixer with a second capacitor. In another aspect, the mixer comprises a diode coupled to ground. In another aspect, the mixer comprises a passive quadrature mixer. In another aspect, the mixer comprises: a first resistor coupled between a voltage source and a mixer output; an inductor coupled between the mixer output and a mixer input; and a diode coupled between the mixer input and a ground. In another aspect, the baseband amplifier comprises: a second resistor coupled between a voltage source and a negative input of an amplifier; a third resistor and a fourth capacitor connected in parallel between the negative input of the amplifier and a ground; a fourth resistor coupled between a mixer output and a positive input of the amplifier; and a fifth resistor and a third capacitor connected in parallel between the positive input of the amplifier and an output of the amplifier. In another aspect, a gain block amplifier is coupled to the low noise amplifier, and a power divider is coupled between the gain block amplifier and the mixer. In another aspect, the power divider separates a signal received by the microwave receiver into a LO signal and a RF signal. In another aspect, an analog to digital converter is coupled to the baseband amplifier, and a processor or computer is coupled to the analog to digital converter. In another aspect, the microwave passive sensor is not synchronized or cooperative with the microwave source. In another aspect, the microwave source comprises a Wi-Fi access point or a Bluetooth signal source. In another aspect, the microwave passive sensor provides Doppler information of a moving target based on reflected signals from the moving target and direct-path signals from the microwave source. In another aspect, the moving target comprises a human, an animal, an object, a fluid, a human activity, a human gesture, a human vital sign or an animal vital sign. In another aspect, the microwave passive sensor is tunable to different frequencies or scans a range of frequencies.

Note that the invention is not limited to the embodiments described herein, instead it has the applicability beyond the embodiments described herein. The brief and detailed descriptions of this disclosure are given in the following.

### BRIEF DESCRIPTION OF THE DRAWINGS

For a more complete understanding of the features and advantages of the present invention, reference is now made to the detailed description of the invention along with the accompanying figures and in which:

FIG. 1 is a block diagram of a conventional Doppler radar in accordance with the prior art;

FIG. 2 is a block diagram of a microwave passive sending technique in accordance with one embodiment of the present disclosure;

FIG. 3 is the experimental setup in accordance with one embodiment of the present disclosure;

FIG. 4 is a microwave passive sensing device in accordance with one embodiment of the present disclosure;

FIG. 5 is diagram showing the power level of the received microwave signals at the input of each RF component for the proposed passive sensing microwave architecture in accordance with one embodiment of the present disclosure;

FIG. 6 is a graph showing the experimental results of the recovered I/Q baseband signals in accordance with one embodiment of the present disclosure;

FIG. 7 is a graph showing the experimental results of the spectra of the recovered I/Q baseband signals in accordance with one embodiment of the present disclosure;

FIG. 8 is a diagram of a microwave passive sensing device in accordance with another embodiment of the present disclosure;

FIG. 9 is a circuit diagram for a passive Doppler radar in accordance with another embodiment of the present disclosure;

FIG. 10A is a circuit diagram for a full small-signal model for a diode-based mixer in accordance with one embodiment of the present disclosure;

FIG. 10B is a circuit diagram for a high-frequency RF model a diode-based mixer in accordance with one embodiment of the present disclosure;

FIG. 10C is a circuit diagram for a low frequency (baseband) model for a diode-based mixer in accordance with one embodiment of the present disclosure;

FIG. 11 is graph depicting a diode-based mixer's conversion ratio versus bias voltage from SPICE simulation and calculation in accordance with one embodiment of the present disclosure;

FIG. 12A is a diagram of a circuit under test for a diode-based mixer stand-alone test in accordance with one embodiment of the present disclosure;

FIG. 12B is a graph depicting S11 for the diode-based mixer versus voltage in accordance with one embodiment of the present disclosure;

FIGS. 13A-13F are graphs depicting a diode-based mixer's down-conversion ratio with different input power levels and different bias voltages in accordance with one embodiment of the present disclosure;

FIG. 14A is a photograph for the PCB of the passive radar for an experiment with an actuator as a target in accordance with one embodiment of the present disclosure;

FIG. 14B is a photograph of a laboratory setup for an experiment with an actuator as a target in accordance with one embodiment of the present disclosure;

FIG. 14C is graph depicting a transient waveform for the baseband output in the experiment of FIGS. 14A-14B in accordance with one embodiment of the present disclosure;

FIG. 14D is a graph depicting a spectrum for the baseband output in the experiment of FIGS. 14A-14B in accordance with one embodiment of the present disclosure;

FIG. 15A is a photograph of a laboratory setup for an passive radar experiment with a human subject in accordance with one embodiment of the present disclosure;

FIG. 15B is graph depicting a raw data for the passive radar baseband in the experiment of FIG. 15A in accordance with one embodiment of the present disclosure;

FIG. 15C is graph depicting a Fast Fourier Transform (FFT) of the high-pass filtered passive radar's output and fingertip pulse monitor's output in the experiment of FIG. 15A in accordance with one embodiment of the present disclosure;

FIG. 15D is graph depicting a FFT of the low-pass filtered passive radar's output and the chest band respiration monitor's output in the experiment of FIG. 15A in accordance with one embodiment of the present disclosure;

FIG. 16A is a photograph illustrating a hand-click in a hand-gesture detection in accordance with one embodiment of the present disclosure;

FIG. 16B is a photograph illustrating a horizontal rotation of a hand in a hand-gesture detection in accordance with one embodiment of the present disclosure;

FIG. 16C is a spectrogram for a hand-click in a hand-gesture detection in accordance with one embodiment of the present disclosure;

FIG. 16D is a spectrogram for a horizontal rotation of a hand in a hand-gesture detection in accordance with one embodiment of the present disclosure;

FIG. 17 is a block diagram of a system in accordance with one embodiment of the present disclosure; and

FIG. 18 is a flow chart of a method for passively detecting a movement of a target in accordance with one embodiment of the present disclosure.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity but include the general class of which a specific example may be used for illustration. The terminology herein is used to describe specific embodiments of the invention, but their usage does not limit the invention, except as outlined in the claims.

Various methods are described below to provide an example of each claimed embodiment. They do not limit any claimed embodiment. Any claimed embodiment may cover methods that are different from those described above and below. The drawings and descriptions are for illustrative, rather than restrictive, purposes.

Microwave architectures for passive sensing applications are disclosed herein. The capability to simultaneously retrieve both the transmitted signal from a non-cooperative microwave source and the signals scattered by a target is the key to enable the identification of Doppler frequencies associated with the target of interest. Neither hardware modification nor synchronization to the signal source is needed, and a simple Fourier-based analysis of the baseband responses is used to extract the Doppler information of a moving target. As a result, the radio-frequency topology leverages current and next generation Wi-Fi, Bluetooth, and wireless power transfer infrastructure to make best use of RF radiations, spectrum, and wireless networks for ubiquitous smart home, health care, and smart living by passively using ambient RF signals for the identification of a target's motion.

Moreover, the passive sensing applications disclosed herein enable the possibility of microwave passive sensing at most places on our planet. Since microwave passive sensing does not transmit its own radio frequency (RF) signals, it inherently reduces cost and power compared with conventional radar sensors. Moreover, it provides a solution to address the interference with the wireless communication signals and improves the spectrum efficiency.

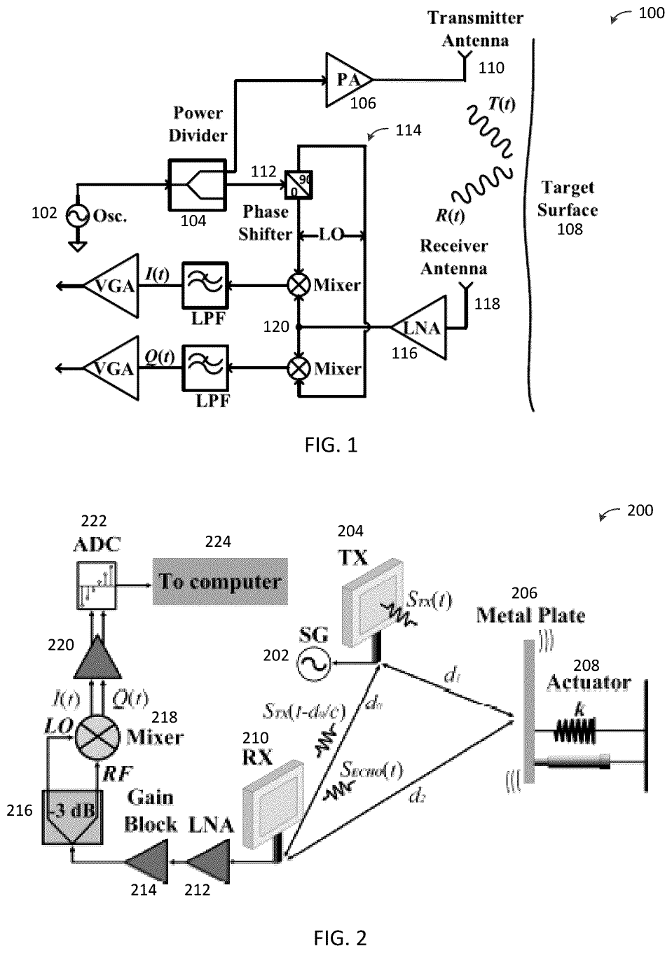

As previously described in reference to FIG. 1, a single-tone radio-frequency (RF) signal is divided in two paths in a typical Doppler transceiver. One is transmitted towards a moving target, while the other is sent to the local oscillator (LO) port of an RF mixer in the receiver chain. Part of the backscattered RF signals, which has the target's motion information modulated in phase, is captured by the receiving antenna. After amplification, the captured received signal is directly sent to the second input port of a RF mixer, namely the RF port, so it can be down-converted to baseband. In contrast, the passive sensing devices in accordance with the present disclosure does not have an RF transmitter and does not use any dedicated reference signal path to recover the transmitted signal.

Two embodiments of the present disclosure will be described. The first embodiment is a single receiver passive radar without an injection-locking oscillator. The second embodiment is a passive radar with a customized diode-based single-input mixer.

Now referring to FIG. 2, a block diagram for a microwave passive sensing technique 200 in accordance with one embodiment of the present disclosure is shown. The transmitted signals produced by an active microwave signal source 202 such as Wi-Fi access points or a Bluetooth signal source can be leveraged. A signal generator (SG) operating at 2.4-GHz and a transmitter 204 were employed as the active microwave signal source 202. Some of the transmitted signals (S_{TX}(t)) are phase-modulated by the target's motion, which is produced by a metal plate 206 that moves periodically with the help of a mechanical actuator 208. The nominal distances between the TX antenna 204 and RX antenna 210, between the TX antenna 204 and the metal plate 206, and between the RX antenna 210 and the metal plate 206 are d₀, d₁, and d₂, respectively. Part of the microwave signals (S_{ECHO}(t)) is backscattered towards the passive sensing device, where it is mixed with a delayed version of the transmitted signal (S_{TX}(t−d₀/c)) and with itself. The normalized baseband signals can be mathematically expressed as I(t)=cos(4πx(t)/λ+θ) and Q(t)=sin(4πx(t)/λ+θ), where θ=4π(−d₀+d₁+d₂/λ is the phase delay associated with the direct transmission path, and the phase delays due to the TX antenna-metal plate path and metal plate-RX antenna path. A is the wavelength of the transmitted signal S_{TX}(t), and x(t)=m sin(ω₀t) is the mechanical motion of the metal plate 206 with m and ω₀ being the motion's amplitude and frequency, respectively. Therefore, the metal plate's motion can be estimated by analyzing the recovered baseband I/Q responses in the time and frequency domains. As shown, the passive sensing device includes the RX antenna 210, low noise amplifier 212, gain block amplifier 214, power divider 216, mixer 218 and baseband amplifier 220, which is coupled to an analog-to-digital converter 222 and computer 224. Note that other frequencies can be uses and 2.4-GHz is merely provided as a non-limiting example.

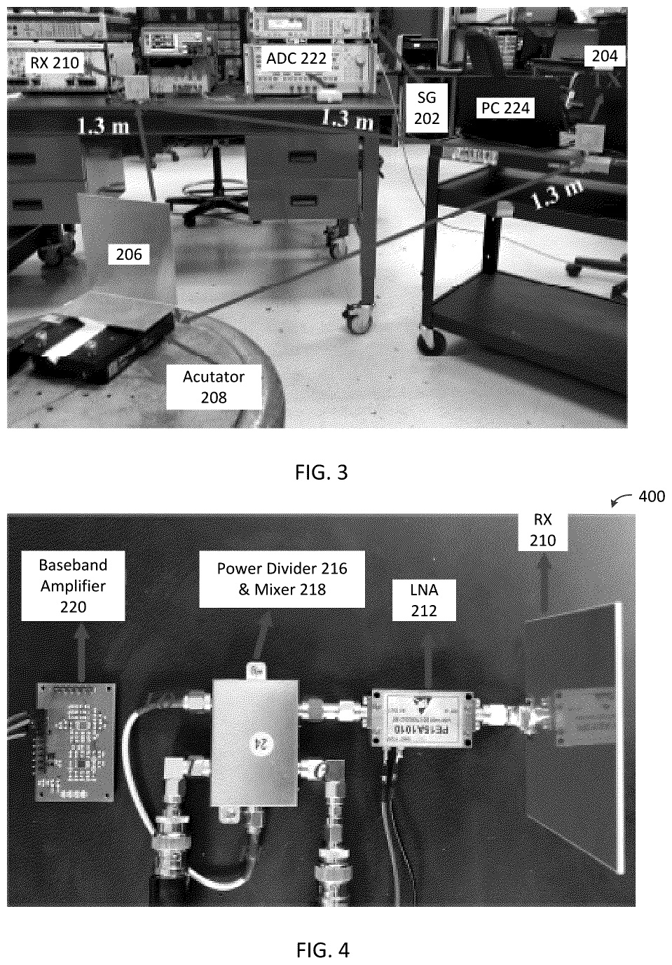

To validate the feasibility of the passive sensing microwave architecture, experiments were conducted to measure the mechanical motion of an actuator (Zaber T-NAO8A50) 208 using a 2.4-GHz microwave passive sensing device (MPSD) as depicted in FIG. 3. A rectangular metal plate 206 with dimensions of 10 cm×10 cm was attached to the actuator 208. The distance between the MPSD and the metal plate 206 was approximately 1.3 m. The distance between MPSD and the active microwave signal source, i.e., a signal generator (SG) 202 operating at 2.4-GHz, was 1.3 m. A microwave cable connected the SG 202 to a 2.4-GHz patch antenna (TX) 204. The RF output power at the input of the TX antenna 204 was measured as 8 dBm. The distance between the TX antenna 204 and the metal plate 206 was 1.3 m. The gain of the TX/RX patch antennas (204 and 210) are 5.8 dBi. The actuator 208 was programmed to produce a periodic sinusoidal movement of 1 Hz with a peak-to-peak amplitude of 1 mm to evaluate the device's sensitivity.

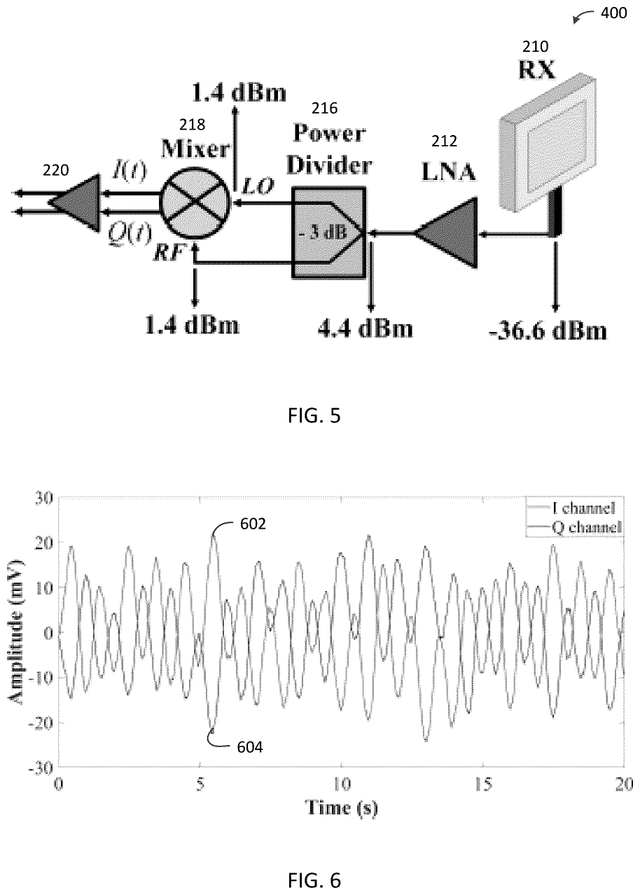

Referring now to FIG. 4, a microwave passive sensing device 400 in accordance with one embodiment of the present disclosure is shown. A 41-dB low-noise amplifier (LNA) 212 designed by Pasternack (PE15A1010) was placed at the RF front end of the device 400. Its noise figure and its input referred P1 dB are −25 dBm and 0.9 dB at 2.4-GHz, respectively. Although the relatively high-gain LNA 212 boosted the weak signals reflected by the moving target, its wideband input matching (2-6 GHz) also allowed the coupling of other undesired signals, which contributes a possible increase in the noise floor. The received signal is then sent to a demodulator module, which consists of a power divider 216 and a RF mixer 218. The power divider 216 immediately follows the LNA 212 and allows the separation of the received signals in the LO and RF signals, which are the inputs of the RF mixer 218. The RF mixer 218 used in this work is a passive quadrature mixer optimized to operate at 2.4-GHz. A signal of at least 1 dBm should be applied to the LO port of the passive mixer, which also explains the use of the high-gain LNA 212. The power level of the received microwave signals at the input of each RF component of the passive sensing device 400 is exhibited on FIG. 5. Finally, the quadrature I/Q baseband signals are further amplified by a baseband amplifier 220 with a baseband gain of 100 V/V. The baseband outputs were digitized using a 16-bit analog-to-digital converter (ADC) DI-2108 designed by DATAQ. The sampling frequency was 200-Hz.

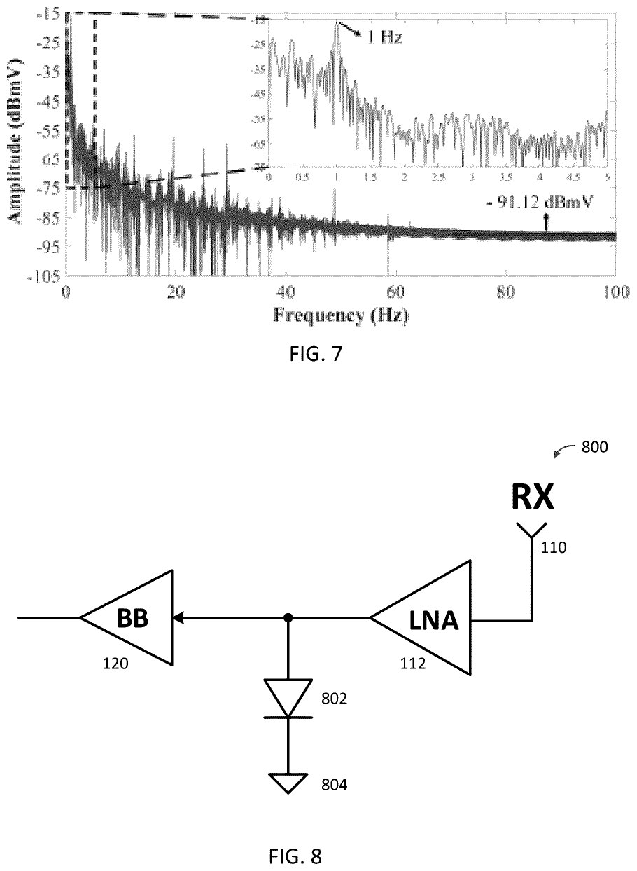

Now referring to FIG. 6, the recovered I/Q baseband signals (I channel (red) 602 and Q channel (blue) 604) are shown, after applying a moving-average approach along each channel in accordance with one embodiment of the present disclosure. Each mean is calculated over a sliding window of length 100 elements across neighboring elements of each baseband channel. This smooths the received signal and eliminates high-frequency random interferences coupled to the passive microwave system. It should be noted that the device was able to successfully detect the moving metal plate as shown in FIG. 6. In FIG. 7, the corresponding spectra of the recovered I/Q baseband signals is illustrated. The fundamental frequency of the target's motion (1 Hz) is highlighted. The noise floor level was obtained by averaging the spectral power over frequencies higher than 70 Hz. It was estimated as −91.12 dBmV as highlighted on FIG. 7, while the power level of the fundamental tone was estimated as −16 dBmV, which confirms the effectiveness of the proposed fully passive microwave architecture for the detection of low-amplitude motions.

In the passive radar architecture, a third-party transmitter is responsible for transmitting the RF signal T(t) to the target.

$$T ( t ) = {V}_{TX} cos ( 2 π ft + φ ( t ) ) , \qquad ( 1 )$$

where f is the transmitted signal's frequency, V_{TX} is the amplitude of the transmitted signal and φ(t) is the phase noise of the transmitted signal. The RX antenna of the passive radar picks up the reflected signal from the target R₁(t).

$${R}_{1} ( t ) = {V}_{1} cos ( 2 π f ( t - {t}_{a} ( t ) ) + φ ( t - {t}_{d} ) ) , \qquad ( 2 )$$

where t_{d}(t) is the time delay between the transmitted signal and the received signal, and V₁ is the amplitude of the received signal. The target's movement results in variation in the time delay and modulates the phase of the signal.

If the passive radar is on the line of sight path from the transmitter, the RX antenna also picks up the transmitted signal directly. If the receiver is not on the line of sight, static clutters will reflect the transmitted signal to the receiver, and the RX antenna will pick up the static clutter reflected signal. The received signals have a constant phase in both cases because the transmitter, the receiver, and the static clutters are all stationary. The directly received signal from the transmitter can be written as

$${R}_{2} ( t ) = {V}_{2} cos ( 2 π ft + {φ}_{0} + φ ( t - {t}_{do} ) ) , \qquad ( 3 )$$

where V₂ is the received signal's amplitude, φ₀ is the constant phase shift, and t_{do} is a constant time delay between the transmitted signal and this signal. Note that the phase noise term (t−t_{d}) in (2) and (t−t_{do}) in (3) are still correlated. Therefore, the difference between these two terms is small and negligible in short-range applications according to the range correlation theory [15], [29], and [31].

It is generally true to claim that the received signal always contains the directly transmitted signal without phase modulation and the phase-modulated signal reflected from the moving target in a natural environment. If one appreciates the fact that the passive radar always receives both signals, even though both of their amplitudes can vary independently, the target can be detected by multiplying the transmitted signal with the reflected signal. Any non-linear device can achieve the multiplication with the square term for the sum of those two signals. The proposed architecture uses a diode-based single-input mixer design since a diode has an exponential relationship between its current and voltage, which is highly non-linear. From [16], the diode current I(t) with a small-signal approximation is given below

$$I ( t ) = {I}_{0} + {g}_{m} ( {R}_{1} ( t ) + {R}_{2} ( t ) ) + \frac{{g}_{m}^{′}}{2} {( {R}_{1} ( t ) + {R}_{2} ( t ) )}^{2} + ⋯ , \qquad ( 4 )$$

where I₀ is the bias current, gₘ=dI²/d²V is the transconductance, and is the second-order derivative of the current applying on the square term of the input voltage. The square term in the current is the main non-linear contribution to multiply and down-convert the signals. The baseband output current I_{BB}(t) can be found by substituting (2) and (3) into the square tem inside (4), and then using trigonometric identities to expand it.

$${I}_{BB} ( t ) = \frac{{g}_{m}^{′}}{2} {V}_{1} {V}_{2} cos ( 2 {π ft}_{d} ( t ) + {φ}_{0} ) . \qquad ( 5 )$$

To extract the target movement from (5), the Fast Fourier Transform (FFT) will be applied, which is the same approach used to process signals obtained by conventional Doppler radar [17].

Referring now to FIG. 8, a diagram of a microwave passive sensing device 800 in accordance with one embodiment of the present disclosure is shown. In this embodiment, the power divider 216 and mixer 218 of FIG. 4 are replaced with a diode 802 coupled to ground 804. This embodiment has fewer components than the embodiment shown in FIG. 4, which results in a smaller footprint, lower cost and lower DC power requirements. Moreover, the higher conversion gain for the diode mixer provides better sensitivity. The peak conversion gain for the diode mixer is about +8 dB, which is more than a 30 dB improvement over the other design.

A passive radar will now be described for the 2.4 GHz Wi-Fi frequency band with a customized diode-based single-input mixer to maximize the down-conversion gain in accordance with another embodiment of the present disclosure. This architecture has a low cost because it only includes an RF gain stage, a diode-based mixer [18]-[26], and a baseband amplifier as the main components.

It is worth mentioning that the passive radar for Wi-Fi can be treated as a bistatic radar with a third-party transmitter and a customized receiver [27]. For a bistatic radar, if the target movement is perpendicular to the line of bistatic radar, it will not generate sufficient Doppler frequency. For biomedical vital signs monitoring, the chest movement for respiration and heartbeat is in all directions [28]. Therefore, the vital signs can be detected regardless of the relative location to the passive radar. Nevertheless, the placement of the passive radar plays a critical role in the strength and quality of the received signals.

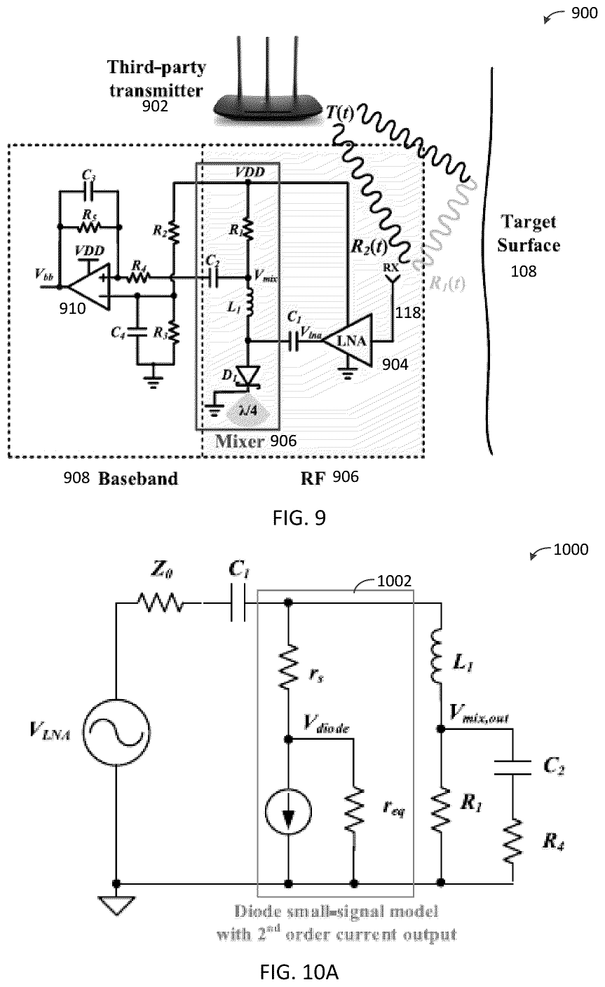

Referring now to FIG. 9, a circuit diagram 900 for a passive Doppler radar in accordance with another embodiment of the present disclosure is shown. One or more third-party transmitters 902 transmit signals T(t). The received signals R₁(t) and R₂(t) are received by the receiver antenna 118 and amplified by a LNA 904, which is implemented as two LNAs (ADL5611) cascaded to provide a 40 dB gain for microwave passive sensing applications. The LNA's output Vₗₙₐ is AC coupled to a Schottky diode D₁'s anode (Infineon-BAT15-02LRH) with a capacitor C₁ to isolate the output common-mode of the LNA from the diode's bias voltage. The diode D₁ is used as a single port mixer 906. The anode of D₁ is also connected to an inductor L₁ (Coilcraft_0603HP_R36) as an RF choke. The other side of L₁ is connected to a resistor R₁, then to the supply VDD. R₁ needs to meet the power dissipation requirement since it carries a large current. The output of the diode-based mixer 906 is between L₁ and R₁, and it is AC coupled to the baseband amplifier 908 by a capacitor C₂. The cathode of D₁ is shorted to a DC ground and a radius stub which works as an AC ground at 2.4 GHz. The baseband amplifier 908 has an amplifier 910 with a low-pass filter (LPF) implemented by R₂, R₃, and C₄ for the common-mode reference to filter out the supply noise. It is also designed to have a band-pass transfer function for the input signal. The lower and upper frequencies for the passband are set by 1/(2πC₂R₄) and 1/(2πC₃R₅), respectively. Since the frequency range for the signal of interest is from sub-Hz to hundreds of Hz, the high-pass corner frequency is set at around 0.1 Hz to remove the DC offset at the mixer's output, and the low-pass corner frequency is set around 1 kHz and used as an anti-aliasing filter before digitizing the signal with an analog-to-digital converter. The DC gain of the amplifier 910 is set by the ratio of R₅ and R₄. The output of the amplifier 910 is V_{bb}.

The purpose of having the RF choke L₁ is to provide a high impedance at 2.4 GHz so that the load impedance on the other side of L₁ is negligible when it is compared with the impedance of L₁ itself. The resistor R₁ is required to provide an impedance between D₁ and supply at the baseband frequency, such that the baseband current from D₁ can flow through it to generate an output voltage. Otherwise, L₁ is a short at the baseband frequency and the supply shorts directly to the anode. Since the resistance of R₁ is in parallel with the small-signal resistance of the diode, it needs to be much larger than the small-signal impedance of the diode to not affect the down-conversion gain of the diode-based mixer. Another function of R₁ is to set the bias current for the diode. Since D₁ is a Schottky diode, the voltage across the diode is approximately zero, and the bias current of the diode is approximately as I₀≈Vdd/R₁. The diode I-V relationship is given by V>>nkT/q. From (6), the diode's small-signal resistance r_{eq} and g′ₘ can be calculated as follows,

$$I = {I}_{s} ( {e}^{\frac{q V}{nkT}} - 1 ) \approx {I}_{s} {e}^{\frac{q V}{nkT}} , \qquad ( 6 )$$

where q is the charge of an electron, k is Boltzmann's constant, T is temperature, n is the ideality factor which typically varies from 1 to 2 based on the structure of the diode, and I_{S} is the saturation current. The approximation holds for

$${r}_{eq} {\vert}_{I = {I}_{0}} = \frac{1}{{g}_{m}} {\vert}_{I = {I}_{0}} = {( \frac{dI}{d V} )}^{- 1} {\vert}_{I = {I}_{0}} = \frac{1}{\frac{q}{nkT} {I}_{0}} . \qquad ( 7 )$$

$${g}_{m}^{′} {\vert}_{I = {I}_{0}} = \frac{{d}^{2} I}{{d V}^{2}} {\vert}_{I = {I}_{0}} = {( \frac{q}{nkT} )}^{2} {I}_{0} . \qquad ( 8 )$$

Equations (7) and (8) shows that the bias current of the diode determines both r_{eq} and g′ₘ, and they differ from each other just by a constant. Both of them contribute to the diode's down-conversion gain, as will be shown later.

As mentioned previously, the RF choke provides a high impedance at 2.4 GHz. Hence, the total impedance looking into the diode is mainly decided by the diode's small-signal resistance, assuming that the parasitic capacitor at 2.4 GHz is also negligible. Hence, the bias current of the diode also affects the input impedance matching for the mixer and indirectly affects the diode's down-conversion gain. Since r_{eq} is inversely proportional to the bias current, it drops from infinite to zero as the bias current increases from 0 to infinite. There is a bias point that makes the diode's small-signal resistance equals to the output impedance of the LNA 904 and achieves good matching. On the other hand, the g′ₘ keeps increasing as the current increases. Because of this, the best bias point for the maximum down-conversion gain may not be the bias point that allows the best matching.

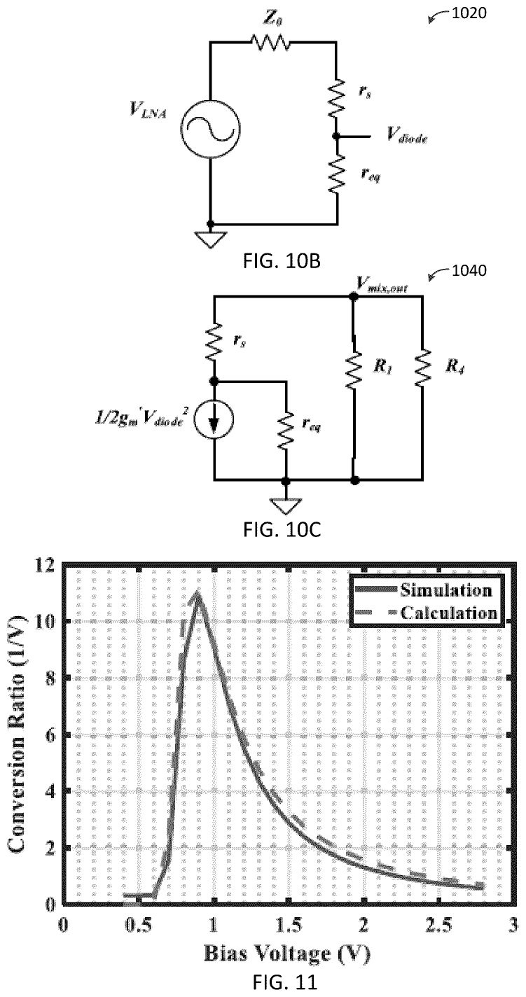

The previous analysis shows that all the parameters that affect the down-conversion gain are related to the bias current of the diode. Hence, an equation that relates the bias current to the down-conversion gain can be derived to provide more insight into selecting the bias current that maximizes the down-conversion gain. A small-signal model with the second-order term of the Taylor series for diode current can be developed to analyze the down-conversion gain for the single-input diode-based mixer. A mixer's small-signal model 1000 in accordance with one embodiment of the present disclosure is shown in FIG. 10A. V_{LNA} is the output of the LNA, and Z₀ is 50Ω assuming that the LNA output is matched. The circuits circled in blue (labeled as 1002) are the proposed equivalent model for the diode to facilitate the calculation: rₛ is the series resistance for the diode; r_{eq} is the small-signal resistance of the diode; the current source ½g′ₘV_{diode}² represents the non-linear baseband current which comes from the square term of (4). The parasitic capacitance and inductance of the equivalent model are ignored here for simplicity because they are not dominant at 2.4 GHz. One should note that R₄ is drawn to be connected to a small-signal ground in the model also for simplicity. It is correct at low frequency since the baseband amplifier's inputs are virtual grounds. At high frequency, the gain of the amplifier drops to zero, and it should be treated as open. If the parasitic capacitance is considered, R₄ should be collected to the parasitic capacitance. Since R₄ can be designed to be much larger than r_{eq}, it can be ignored even if it is connected to the ground. Therefore, assuming R₄ is short to ground for all frequencies does not affect the accuracy of the conclusion.

Since the mixer converts high frequency RF signals to low frequency baseband signals, the small-signal model 1000 can be simplified by analyzing the high frequency and low frequency separately. The small-signal models for high frequency 1020 and low frequency 1040 in accordance with on embodiment of the present disclosure are shown in FIG. 10B and FIG. 10C, respectively. It should be noted that the nonlinear current of the diode-based mixer needs to exclude the voltage drop on the series resistance rₛ, so V_{diode} is the voltage applied on the p-n junction of the diode and contributes to the down-conversion. At the high frequency (RF), C₁ and C₂ are short, L₁ is an open, and the current source ½g′ₘV_{diode}² is eliminated since it models the baseband current. At the low frequency (baseband frequency), C₁ is open, L₁ is short, and C₂ is designed to be short at the frequency of the interest for the baseband so that the mixer output can be AC-coupled to the baseband. Note that the V_{diode} in the current source expression ½g′ₘV_{diode}² is the RF input voltage for the mixer, so it needs to be calculated from the high frequency model. From FIG. 10B, it is easy to find the actual RF input voltage to the diode V_{diode} as

$${V}_{diode} = {V}_{LNA} * \frac{{r}_{eq}}{{Z}_{0} + {r}_{s} + {r}_{eq}} . \qquad ( 9 )$$

From the low frequency model, the output of the mixer V_{mix,out} can be calculated with Thevenin equivalent circuit.

$${V}_{mix , out} = \frac{1}{2} {g}_{m}^{′} {V}_{diode}^{2} {r}_{eq} * \frac{{R}_{4} // {R}_{1}}{{R}_{4} // {R}_{1} + {r}_{eq} + {r}_{s}} . \qquad ( 10 )$$

Substituting (9) into (10),

$${V}_{mix , out} = \frac{1}{2} {g}_{m}^{′} {r}_{eq} {V}_{LNA}^{2} \frac{{r}_{eq}^{2}}{{( {Z}_{0} + {r}_{s} + {r}_{eq} )}^{2}} * \frac{{R}_{4} // {R}_{1}}{{R}_{4} // {R}_{1} + {r}_{eq} + {r}_{s}} \qquad ( 11 )$$

Similar as the derivation from (4) to (5), the mixer's baseband voltage output V_{BB} can be written as

$${V}_{BB} = \frac{1}{2} {g}_{m}^{′} {r}_{eq} {V}_{1} {V}_{2} \frac{{r}_{eq}^{2}}{{( {Z}_{0} + {r}_{s} + {r}_{eq} )}^{2}} * \frac{{R}_{4} // {R}_{1}}{{R}_{4} // {R}_{1} + {r}_{eq} + {r}_{s}} \qquad ( 12 )$$

If R₄>>R₁>>(r_{eq}+r), substituting (7) and (8) into (12), V_{BB} can be simplified to

$${V}_{BB} \approx \frac{1}{2} \frac{q}{nkT} {V}_{1} {V}_{2} * \frac{1}{{( \frac{q}{nkT} {I}_{0} ( {Z}_{0} + {r}_{S} ) + 1 )}^{2}} \qquad ( 13 )$$

Equation (13) indicates that the amplitude of the baseband output voltage reduces as the bias current increases. On the other hand, when the I₀ is small, r_{eq}>>R₄, R₁, substituting (7) and (8) into (12) again, V_{BB} can be simplified into a different equation

$${V}_{BB} \approx \frac{1}{2} {( \frac{q}{nkT} )}^{2} {I}_{0} {V}_{1} {V}_{2} ( {R}_{4} // {R}_{1} ) \qquad ( 14 )$$

Equation (14) indicates that mixer output amplitude increases as the bias current increase.

The analysis above shows that an ideal bias point must exist to maximize the down-conversion gain. The down-conversion gain for the diode was simulated with a spice diode model to validate the analysis. Both V₁ and V₂ were set to 10 mV to approximate the small-signal condition. The baseband outputs were recorded with different bias voltages. Then, the same bias points were used to calculate the baseband output using (12). Conventionally, the down-conversion gain of a mixer is defined as G=V_{BB}/V_{RF}In under a fixed LO output power. Because the baseband signal for the single-input mixer is a strong function of both input signals, it might be easier to use the conversion ratio defined as V_{BB}/V₁/V₂ instead of the conversion gain to analyze and compare different mixer designs. The conversion gain can be calculated from the conversion ratio by multiplying the conversion ratio with the LO voltage. FIG. 11 plots the conversion ratio as the function of the bias voltage for both simulation (solid blue line) and calculation (dashed red line). The x-axis is the bias voltage in voltage, and the y-axis is the conversion ratio in 1/V. As can be seen, the calculated conversion ratio matches closely with the spice model simulation.

In this section, the diode-based mixer was characterized stand-alone first to find out the maximum down-conversion gain, and then the full passive radar was tested.

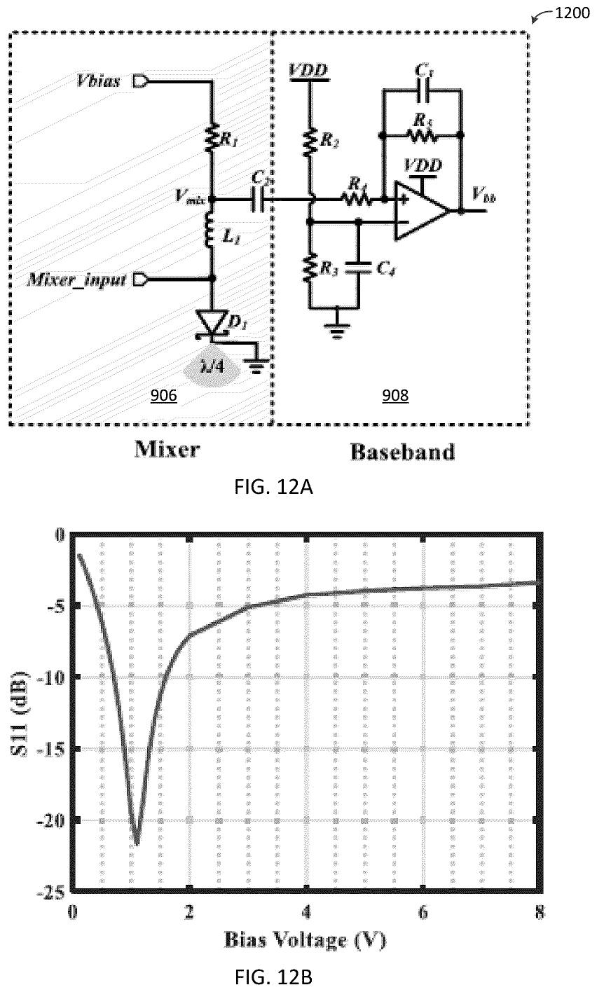

Now referring to FIG. 12A, the circuit 1200 under test (mixer 906 and baseband 908 of FIG. 9) in accordance with one embodiment of the present disclosure is shown. First, the bias voltage for the diode was swept, and the S11 of the diode was measured. FIG. 12B shows the results. The x-axis is the bias voltage in Volts, and the y-axis is the S11 in dB. The best matching was achieved with 1.1 V bias voltage, and the S11 was −21.75 dB.

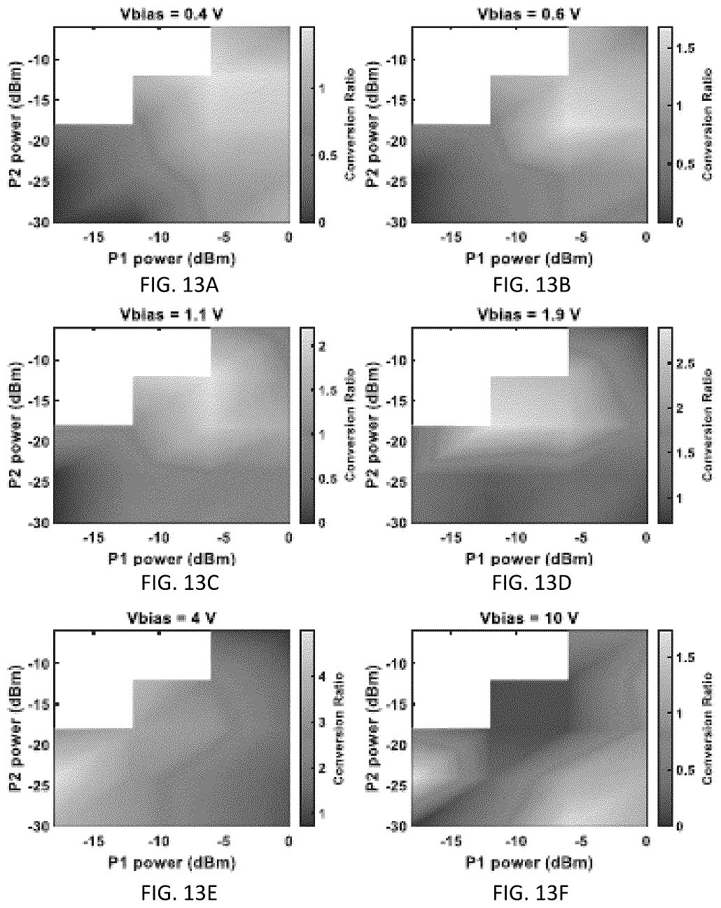

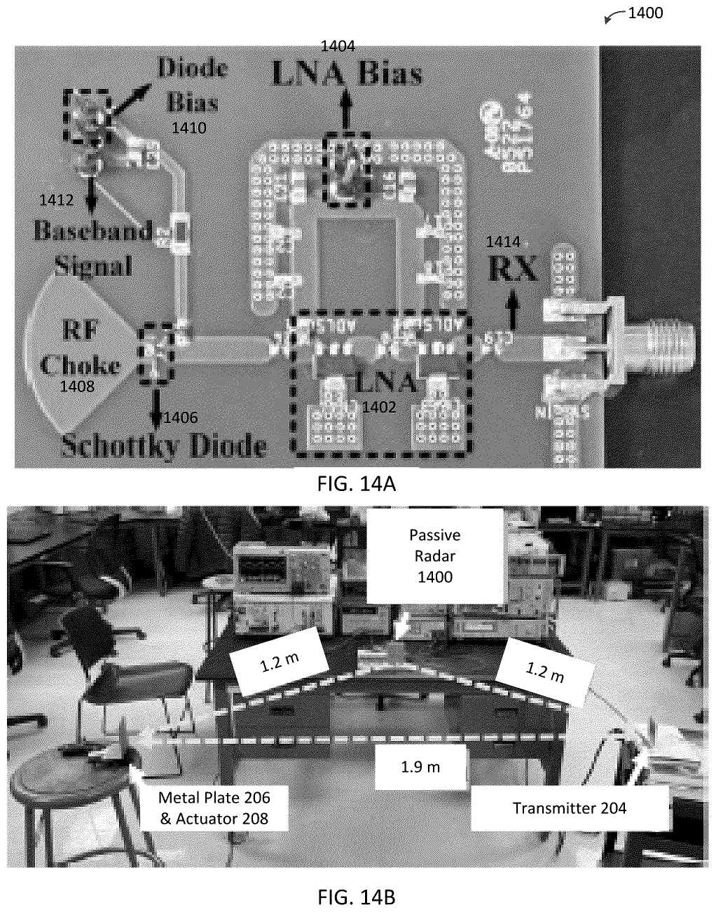

Since the maximum down-conversion gain does not necessarily coincide with a good matching based on the previous analysis, the down-conversion gain of the diode was measured with different bias points in the following experiment. In this experiment, a power combiner was used at the input of the mixer to combine two RF inputs. One RF input was set at 2.4 GHz, and another was set at 2.4 GHz plus 1 Hz offset to simulate the heartbeat's frequency. A few bias points were picked to cover both sides of the best matching point based on FIG. 12B, and the powers of both inputs were swept independently. The baseband amplifier had a voltage gain of 40 dB, and the baseband output voltage at 1 Hz was recorded to calculate the down-conversion ratio. The results were plotted in FIGS. 13A-13F. Each plot is for different bias voltages. The x-axis is the first input's power in dBm; the y-axis is the second input's power in dBm; the color code (shading) represents the down-conversion ratio. The plot shows that when the bias voltage is 4 V, the down-conversion ratio is larger compared with other bias voltages across different combination of two inputs. The maximum down-conversion ratio of 4.985, or 13.95 dB, was achieved. From FIG. 12B, the S11 with a 4 V bias point is about −4.5 dB, which is not an outstanding good matching but is sufficient for the proposed application as the corresponding insertion loss is about 2 dB. Therefore, for the rest of the works on system demonstration, 4-V bias was used.

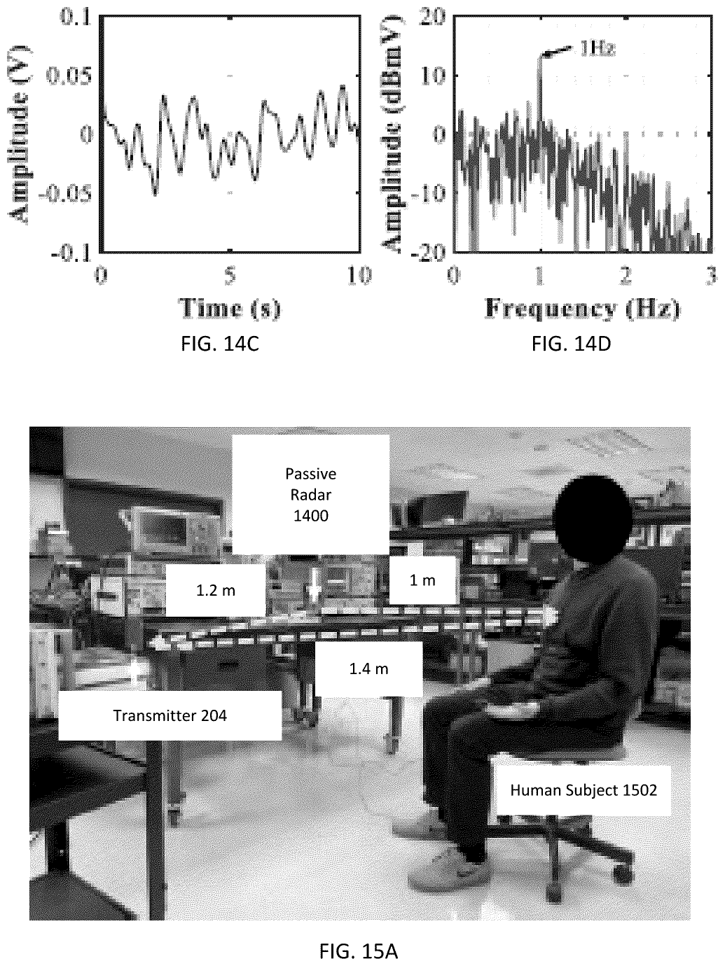

The experiments with the passive radar will now be described. The printed circuit board (PCB) photograph for the passive radar 1400 in accordance with one embodiment of the present disclosure is shown in FIG. 14A. As previously described, the passive radar 1400 include a LNA 1402, LNA bias 1404, Schottky diode 1406, RF choke 1408 and diode bias 1410 to produce a baseband signal 1412 from a received signal 1414. First, an actuator 208 carrying a metal plate 206 (10 cm×10 cm) was used as the target to better control the target movement and get repeatable results. The experiment setup is shown in FIG. 14B. The distance from the transmitter 204 to the passive radar 1400 was 1.2 m, from the transmitter 204 to the actuator 208 was 1.9 m, and from the passive radar 1400 to the actuator 208 was 1.2 m. Because the maximum allowable power for Wi-Fi is 20 dBm, the power level of the transmitted signal was set to 15 dBm in this experiment. The actuator 208 was moving periodically at 1 Hz with 0.2 mm displacement to mimic the chest movement from the human subject's heartbeat. The baseband outputs were digitized using a data acquisition device (DAQ). FIG. 14C shows the transient waveform for the recorded baseband output, the x-axis is the time in seconds, and the y-axis is the amplitude in Volts. FIG. 14D shows the spectrum for the baseband output, the x-axis is the frequency in Hz, and the y-axis is the amplitude in dBmV. A 1 Hz tone is shown in the spectrum which matches the actuator's displacement frequency.

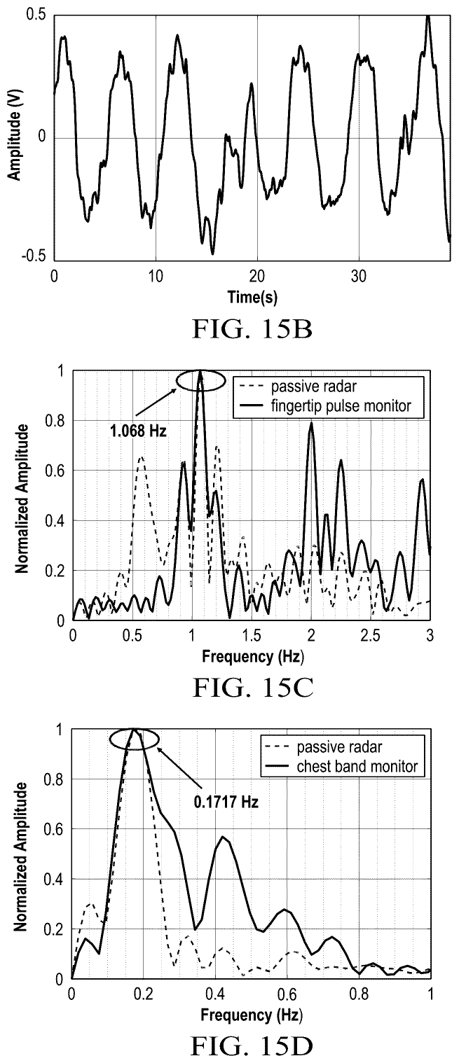

Then, the passive radar was used to measure a human subject's respiration rate and the heart rate. FIG. 15A shows the experiment setup. A human subject 1502 was sitting in front of the radar 1400. At the same time, a fingertip pulse monitor and a chest band monitor were used as the ground truth for the heart rate and respiration, respectively. The distance from the transmitter 204 to the passive radar 1400 was 1.2 m, from the transmitter 204 to the human subject 1502 was 1.4 m, and from the passive radar 1400 to the human subject 1502 was 1 m. The DAQ recorded the baseband output from the passive radar 1400 and the monitors' outputs for the fingertip pulse monitor and the chest band monitor. The transmitter 204 output power was the same 15 dBm as the previous experiment. FIG. 15B shows the transient waveform for the passive radar's baseband output, the x-axis is the time in seconds, and the y-axis is the amplitude in Volts. The digitized baseband output was fed into two different digital filters to separate the heartbeat signals from the respiration signals. For heart rate measurement, a 0.6 Hz high-pass filter was used; for respiration rate measurement, a 0.6 Hz LPF was used. FIG. 15C shows the spectrum of the high-pass filtered passive radar's output (dashed red line) and the spectrum of the fingertip pulse monitor's output for the heartbeat measurement (solid blue line). The x-axis is the frequency in Hz, and the y-axis is the normalized amplitude. FIG. 15D shows the spectrum of the low-pass filtered passive radar's output (dashed red line) and the spectrum of the chest band respiration monitor's output (solid blue line), the x-axis is the frequency in Hz, and the y-axis is the normalized amplitude. It shows that the human subject's heart rate was approximately 64 beats/minute, and the respiration rate was about 10.3 breaths/minute. Both the heart rate and respiration rate measurement results were in agreement with the fingertip pulse monitor's result and the chest band monitor's result, respectively.

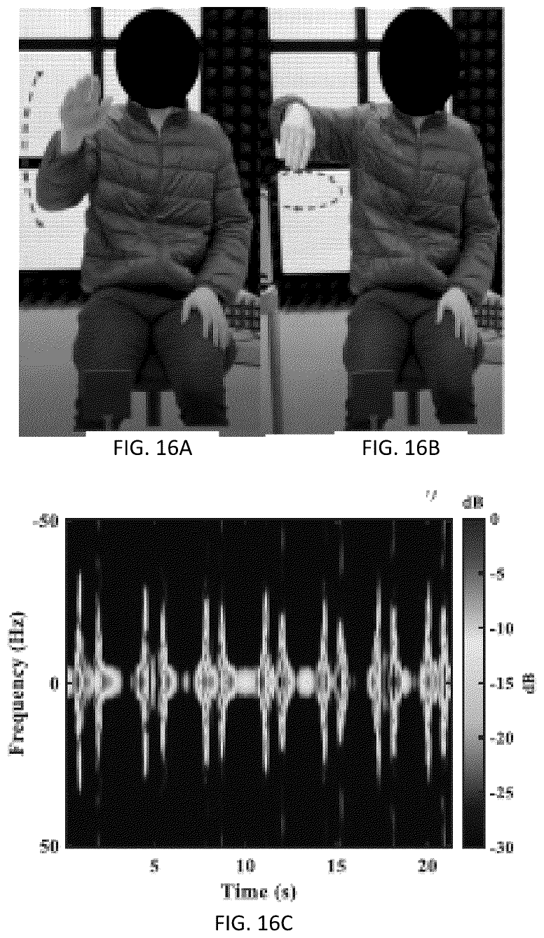

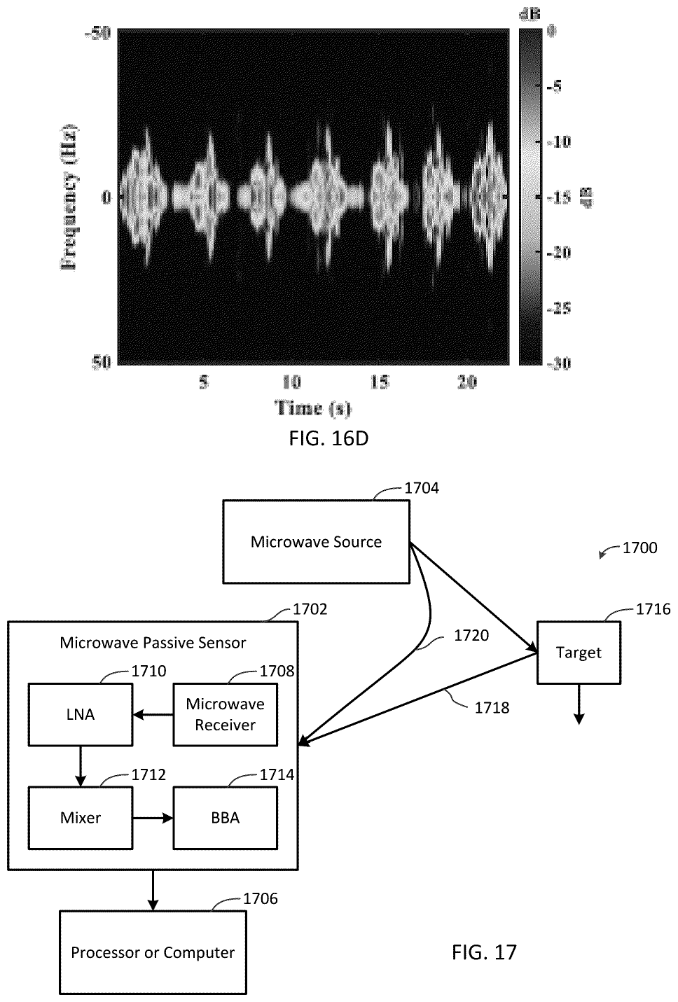

The last experiment was hand gesture detection using micro-Doppler effects. The distance from the transmitter to the passive radar, from the transmitter to the human subject, and from the passive radar to the human subject were all 1 m. FIGS. 16A and 16B illustrate two hand gestures from the human subject in the experiments: hand-click (moving one of the forearms from 90-degree to 0-degree with respect to the ground) and horizontal rotation of the hand. In the experiment, each gesture was repeated for seven times. FIG. 16C presents the Doppler spectrogram for the hand-click. It has two strips for each repetition with a similar profile. The first strip is for the forearm to move down, and the second strip is for the forearm to move back to the original position and prepare for the next repetition. FIG. 16D presents the Doppler spectrogram for the horizontal rotation of the hand. The frequency changes gradually from 0 to a peak and then comes back to 0 because of the circular movement.

A low cost passive radar with a single-input mixer based on a diode for physiological motions of human subjects has been described. The passive radar makes use of the ambient electromagnetic wave illuminated by a third party 2.4 GHz transmitter to achieve target detection, and it does not require to know any other information for the transmitter. A PCB prototype was fabricated with FR-4 substrate and tested in the laboratory. The experiment results demonstrated that the maximum down-conversion ratio of the designed diode-based mixer is about 13.95 dB. Vital signs sensing and hand gesture detection were also demonstrated with a human subject to show the effectiveness of the proposed architecture.

Referring now to FIG. 17, a block diagram of a system 1700 in accordance with one embodiment of the present disclosure. The system 1700 includes a microwave passive sensor 1702, a microwave source 1704, and a processor or computer 1706 coupled to the microwave sensor 1702. The microwave passive sensor 1702 comprises a microwave receiver 1708, a low noise amplifier 1710 coupled to the microwave receiver 1708, a mixer 1712 coupled to the low noise amplifier 1710, and a baseband amplifier 1714 coupled to the mixer 1712. The microwave passive sensor 1702 provides Doppler information of a moving target 1716 based on reflected signals 1718 from the moving target 1716 and direct-path signals 1720 from the microwave source 804.

In one aspect, the low noise amplifier 1710 comprises two cascaded low noise amplifiers. In another aspect, the mixer 1712 is radio frequency (RF) coupled to the low noise amplifier 1710 with a first capacitor C₁, and the baseband amplifier 1714 is RF coupled to the mixer 1712 with a second capacitor C₂ (see FIG. 9). In another aspect, the mixer 1712 comprises a diode coupled to ground. In another aspect, the mixer 1712 comprises a passive quadrature mixer. In another aspect, the mixer 1712 comprises: a first resistor R₁ coupled between a voltage source VDD and a mixer 1712 output; an inductor L₁ coupled between the mixer 1712 output and a mixer 1712 input; and a diode D₁ coupled between the mixer 1712 input and a ground (see FIG. 9). In another aspect, the baseband amplifier 1714 comprises: a second resistor R₂ coupled between a voltage source VDD and a negative input of an amplifier; a third resistor R₃ and a fourth capacitor C₄ connected in parallel between the negative input of the amplifier and a ground; a fourth resistor R₄ coupled between a mixer 1712 output and a positive input of the amplifier; and a fifth resistor R₅ and a third capacitor C₃ connected in parallel between the positive input of the amplifier and an output of the amplifier (see FIG. 9). In another aspect, a gain block amplifier is coupled to the low noise amplifier 1710, and a power divider is coupled between the gain block amplifier and the mixer 1712. In another aspect, the power divider separates a signal received by the microwave receiver 1708 into a LO signal and a RF signal. In another aspect, an analog to digital converter is coupled to the baseband amplifier 1714, and a processor or computer coupled to the analog to digital converter. In another aspect, the microwave passive sensor 1702 is not synchronized or cooperative with the microwave source 804. In another aspect, the microwave source 1704 comprises a Wi-Fi access point or a Bluetooth signal source. In another aspect, the moving target comprises a human, an animal, an object, a fluid, a human activity, a human gesture, a human vital sign, an animal vital sign, or anything that moves and reflects electromagnetic signals. In another aspect, the microwave passive sensor 1702 is tunable to different frequencies or scans a range of frequencies.

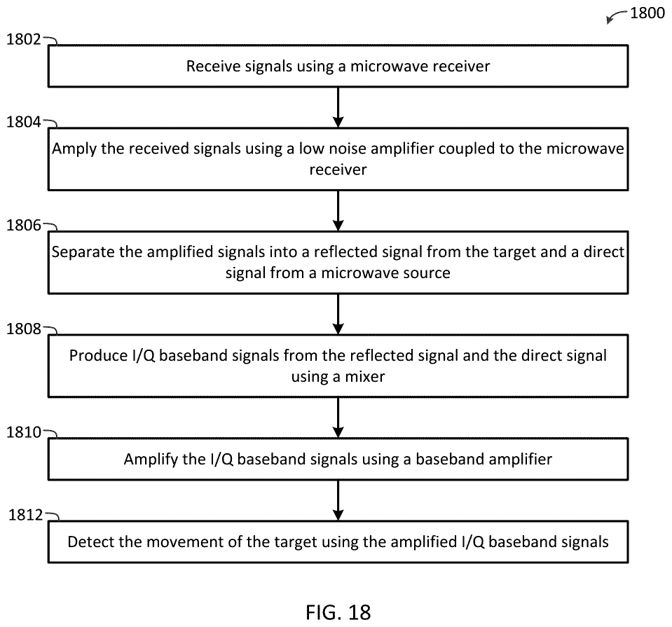

FIG. 18 is a flow chart of a method 1800 for passively detecting a movement of a target in accordance with one embodiment of the present disclosure. The method 1800 includes receiving signals using a microwave receiver in block 1802, amplifying the received signals using a low noise amplifier coupled to the microwave receiver in block 1804, separating the amplified received signals into a reflected signal from the target and a direct signal from a microwave source in block 1806, producing I/Q baseband signals from the reflected signal and the direct signal using a mixer in block 1808, amplifying the I/Q baseband signals using a baseband amplifier in block 1810, and detecting the movement of the target using the amplified I/Q baseband signals in block 1812.

In one aspect, the low noise amplifier comprises two cascaded low noise amplifiers. In another aspect, the mixer is radio frequency (RF) coupled to the low noise amplifier with a first capacitor, and the baseband amplifier is RF coupled to the mixer with a second capacitor. In another aspect, the mixer comprises a diode coupled to ground. In another aspect, the mixer comprises a passive quadrature mixer. In another aspect, the mixer comprises: a first resistor coupled between a voltage source and a mixer output; an inductor coupled between the mixer output and a mixer input; and a diode coupled between the mixer input and a ground. In another aspect, the baseband amplifier comprises: a second resistor coupled between a voltage source and a negative input of an amplifier; a third resistor and a fourth capacitor connected in parallel between the negative input of the amplifier and a ground; a fourth resistor coupled between a mixer output and a positive input of the amplifier; and a fifth resistor and a third capacitor connected in parallel between the positive input of the amplifier and an output of the amplifier. In another aspect, the amplified received signals are further amplified by a gain block amplifier coupled to the low noise amplifier, and the amplified received signals are separated by a power divider coupled between the gain block amplifier and the mixer. In another aspect, the movement of the target is detected by performing a Fourier-based analysis of the amplified I/Q baseband signals using a processor or computer. In another aspect, the method further comprises providing an analog to digital converter coupled to the baseband amplifier, and providing a processor or computer is coupled to the analog to digital converter. In another aspect, the microwave receiver is not synchronized or cooperative with the microwave source. In another aspect, the microwave source comprises a Wi-Fi access point or a Bluetooth signal source. In another aspect, the method further comprises providing Doppler information of a moving target based on reflected signals from the moving target and direct-path signals from a microwave source. In another aspect, the moving target comprises a human, an animal, an object, a fluid, a human activity, a human gesture, a human vital sign, an animal vital sign, or anything that moves and reflects electromagnetic signals. In another aspect, further comprising tuning the microwave passive sensor to different frequencies or scanning a range of frequencies.

A microwave architecture for passive sensing applications has been described herein. By simultaneously injecting the delayed version of the transmitted signals of an RF illuminator and the corresponding phase-modulated signals into the RF and LO ports of the RF mixer, the detection of a 1 mm amplitude motion was successfully achieved, when the proposed device was placed 1.3 m away from the active microwave source and a vibrating target. Also, no reference channels or high complexity digital signal processing techniques were used in this work.

It will be understood that particular embodiments described herein are shown by way of illustration and not as limitations of the invention. The principal features of this invention can be employed in various embodiments without departing from the scope of the invention. Those skilled in the art will recognize or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited features, elements, components, groups, integers, and/or steps, but do not exclude the presence of other unstated features, elements, components, groups, integers and/or steps. In embodiments of any of the compositions and methods provided herein, “comprising” may be replaced with “consisting essentially of” or “consisting of”. As used herein, the term “consisting” is used to indicate the presence of the recited integer (e.g., a feature, an element, a characteristic, a property, a method/process step or a limitation) or group of integers (e.g., feature(s), element(s), characteristic(s), property(ies), method/process steps or limitation(s)) only. As used herein, the phrase “consisting essentially of” requires the specified features, elements, components, groups, integers, and/or steps, but do not exclude the presence of other unstated features, elements, components, groups, integers and/or steps as well as those that do not materially affect the basic and novel characteristic(s) and/or function of the claimed invention.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skill in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

To aid the Patent Office, and any readers of any patent issued on this application in interpreting the claims appended hereto, applicants wish to note that they do not intend any of the appended claims to invoke paragraph 6 of 35 U.S.C. § 112, U.S.C. § 112 paragraph (f), or equivalent, as it exists on the date of filing hereof unless the words “means for” or “step for” are explicitly used in the particular claim.

For each of the claims, each dependent claim can depend both from the independent claim and from each of the prior dependent claims for each and every claim so long as the prior claim provides a proper antecedent basis for a claim term or element.

### REFERENCES

1. J. Wu, Y. Lu, and W. Dai, “Off-grid compressed sensing for WiFi-based passive radar,” in Proc. IEEE Int. Symp. Signal Process. Inf Technol. (ISSPIT), December 2016, pp. 258-262.
2. C. Feng, W. S. A. Au, S. Valaee, and Z. Tan, “Received-signal-strength based indoor positioning using compressive sensing,” IEEE Trans. Mob. Comput., vol. 11, no. 12, pp. 1983-1993, 2011.
3. T. Yoshida and Y. Taniguchi, “Estimating the number of people using existing wifi access point in indoor environment,” in Proc. Eur. Conf of Comput. Sci. (ECCS), 2015, pp. 46-53.
4. S. Tan and J. Yang, “Wifinger: leveraging commodity wifi for finegrained finger gesture recognition,” in Proc. Int. Symp. on Mobile Ad Hoc Netw. and CompUT. (ACM), 2016, pp. 201-210.
5. W. Wang, A. X. Liu, M. Shahzad, K. Ling, and S. Lu, “Device free human activity recognition using commercial WiFi devices,” IEEE J. Sel. Areas Commun., vol. 35, no. 5, pp. 1118-1131, 2017.
6. X. Wang, C. Yang, and S. Mao, “Phasebeat: Exploiting CSI phase data for vital sign monitoring with commodity WiFi devices,” in IEEE Int. Conf on Distrib. Comput. Syst. (ICDCS), 2017, pp. 1230-1239.
7. Y. Ma, G. Zhou, and S. Wang, “Wifi sensing with channel state information: A survey,” ACM Comput. Surv. (CSUR), vol. 52, no. 3, p. 46, 2019.
8. W. Li, B. Tan, and R. Piechocki, “Passive radar for opportunistic monitoring in e-health applications,” IEEE J. Transl. Eng. Health Med., vol. 6, pp. 1-10, 2018.
9. K. Chetty, G. E. Smith and K. Woodbridge, “Through-the-wall sensing of personnel using passive bistatic WiFi radar at standoff distances,” IEEE Trans. Geosci. Remote Sens., vol. 50, no. 4, pp. 1218-1226, April 2012.
10. Y. Lai, C. Chou, M. Tang, T. Horng and F. Wang, “Finger gesture sensing and recognition using a Wi-Fi-based passive radar,” in IEEE MTT-S Int. Microw. Symp. (IMS), 2019, pp. 293-296.
11. M.-C. Tang, F.-K. Wang and T.-S. Horng, “Vital-sign detection based on a passive WiFi radar,” in IEEE MTT-S Int. Microw. Workshop Ser. on RF and Wireless Technol. for Biomed and Healthcare Appl. (IMWS-BIO), 2015, pp. 74-75.
12. Mu-Cyun Tang, F.-K. Wang and T.-S. Horng, “Human gesture sensor using ambient wireless signals based on passive radar technology,” IEEE MTT-S Int. Microw. Symp. (IMS), 2015, pp. 1-4, doi: 10.1109/MWSYM.2015.7167080.
13. F. Wang, M. Tang, Y. Chiu and T. Horng, “Gesture Sensing Using Retransmitted Wireless Communication Signals Based on Doppler Radar Technology,” in IEEE Trans. Microw. Theory Tech., vol. 63, no. 12, pp. 4592-4602, December 2015.
14. A. Parks, A. Sample, Y. Zhao and J. R. Smith, “A Wireless Sensing Platform Utilizing Ambient RF Energy,” in IEEE Topical Meeting on Wireless Sensors and Sensor Networks (WiSNET) (Austin, TX, 2013).
15. D. Tang, J. Wang, W. Hu, Z. Peng, Y.-C. Chiang and C. Li, “A DC-coupled high dynamic range biomedical radar sensor with fast-settling analog DC offset cancelation,” IEEE Trans. Instrum. Meas., vol. 68, no. 5, pp. 1441-1450, May 2019.
16. D. M. Pozar, “Microwave Engineering”, 4^{th} ed., Wiley, 2011.
17. C. Li, V. M. Lubecke, O. Boric-Lubecke and J. Lin, “A review on recent advances in Doppler radar sensors for noncontact healthcare monitoring,” IEEE Trans. Microw. Theory Tech., vol. 61, no. 5, pp. 2046-2060, May 2013.
18. M. R. Barber, “Noise figure and conversion loss of the Schottky barrier mixer diode,” IEEE Trans. Microw. Theory Techn., vol. 15, no. 11, pp. 629-635, November 1967.
19. G. Kuhnle, H. Mayer, H. Olbrich, W. Steffens, and H. Swoboda, “Low-cost long-range radar for future driver assistance systems,” Auto Technol., vol. 3, no. 4, pp. 76-80, July 2003.
20. V. Issakov, H. Knapp, M. Wojnowski, A. Thiede, and W. Simbuerger, “A 22-39 GHz passive mixer in SiGe:C bipolar technology,” in IEEE MTT-S Int. Microw. Symp. Dig., May 2010, pp. 1012-1015.
21. I. Maestrojuan, I. Ederra and R. Gonzalo, “Fourth-harmonic Schottky diode mixer development at sub-millimeter frequencies,” IEEE Trans. THz Sci. Technol., vol. 5, no. 3, pp. 518-520, May 2015.
22. E. OztWrk et al., “A 120 GHz SiGe BiCMOS monostatic transceiver for radar applications,” in Eur. Microw. Integr. Circuits Conf (EuMIC), September 2018, pp. 41-44.
23. D. Rodriguez and C. Li, “A low-power and low-cost monostatic radar based on a novel 2-port transceiver chain,” in IEEE Topical Conf on Wireless Sensors and Sensor Netw. (WiSNeT), 2020, pp. 9-12.
24. B. Sene, H. Knapp, D. Reiter and N. Pohl, “A Compact Monostatic Transceiver Topology Using a Diode-Based Mixer,” in IEEE Topical Meeting on Silicon Monolithic Integr. Circuits in RFSyst. (SiRF), 2021, pp. 7-10.
25. B. Sene, Daniel Reiter, Herbert Knapp, Nils Pohl, “Design of a cost-efficient monostatic radar sensor with antenna on chip and lens in package,” IEEE Trans. Microw. Theory Techn., vol. 70, no. 1, pp. 502-512, 2022.
26. B. Sene, Daniel Reiter, Herbert Knapp, Nils Pohl, “From Gunnplexer to MMIC: an implementation on a single chip,” IEEE Microw. Mag., vol. 23, no. 1, pp. 48-53, 2022.
27. K. Chetty, G. E. Smith and K. Woodbridge, “Through-the-Wall Sensing of Personnel Using Passive Bistatic WiFi Radar at Standoff Distances,” IEEE Trans. GEOSCI. Remote Sens., vol. 50, no. 4, pp. 1218-1226, April 2012.
28. C. Li, Y. Xiao and J. Lin, “Experiment and Spectral Analysis of a Low-Power KaKa-Band Heartbeat Detector Measuring From Four Sides of a Human Body,” IEEE Trans. Microw. Theory Techn., vol. 54, no. 12, pp. 4464-4471, December 2006.
29. A. D. Droitcour, O. Boric-Lubecke, V. M. Lubecke, J. Lin and G. T. A. Kovacs, “Range correlation and I/Q performance benefits in single-chip silicon Doppler radars for noncontact cardiopulmonary monitoring,” IEEE Trans. Microw. Theory Tech., vol. 52, no. 3, pp. 838-848, March 2004.
30. H.-C. Kuo et al., “A fully integrated 60-GHz CMOS direct-conversion Doppler radar RF sensor with clutter canceller for single-antenna noncontact human vital-signs detection,” IEEE Trans. Microw. Theory Techn., vol. 64, no. 4, pp. 1018-1028, April 2016.
31. M. C. Budge and M. P. Burt, “Range correlation effects on phase and amplitude noise,” Southeastcon '93, Proc., IEEE, Charlotte, NC, 1993, pp. 5 p..

1: A microwave passive sensor, comprising:
a microwave receiver;
a low noise amplifier coupled to the microwave receiver;
a mixer coupled to the low noise amplifier; and
a baseband amplifier coupled to the mixer.

2: The microwave passive sensor of claim 1, wherein the low noise amplifier comprises two cascaded low noise amplifiers.

3: The microwave passive sensor of claim 1, wherein:
the mixer is radio frequency (RF) coupled to the low noise amplifier with a first capacitor; and
the baseband amplifier is RF coupled to the mixer with a second capacitor.

4: The microwave passive sensor of claim 1, wherein the mixer comprises a diode coupled to ground.

5: The microwave passive sensor of claim 1, wherein the mixer comprises a passive quadrature mixer.

6: The microwave passive sensor of claim 1, wherein the mixer comprises:
a first resistor coupled between a voltage source and a mixer output;
an inductor coupled between the mixer output and a mixer input; and
a diode coupled between the mixer input and a ground.

7: The microwave passive sensor of claim 1, wherein the baseband amplifier comprises:
a second resistor coupled between a voltage source and a negative input of an amplifier;
a third resistor and a fourth capacitor connected in parallel between the negative input of the amplifier and a ground;
a fourth resistor coupled between a mixer output and a positive input of the amplifier; and
a fifth resistor and a third capacitor connected in parallel between the positive input of the amplifier and an output of the amplifier.

8: The microwave passive sensor of claim 1, further comprising:
a gain block amplifier coupled to the low noise amplifier; and
a power divider coupled between the gain block amplifier and the mixer.

9: The microwave passive sensor of claim 8, wherein the power divider separates a signal received by the microwave receiver into a LO signal and a RF signal.

10: The microwave passive sensor of claim 1, further comprising:
an analog to digital converter coupled to the baseband amplifier; and
a processor or computer coupled to the analog to digital converter.

11: The microwave passive sensor of claim 1, wherein the microwave passive sensor is not synchronized or cooperative with a microwave source.

12: The microwave passive sensor of claim 11, wherein the microwave source comprises a Wi-Fi access point or a Bluetooth signal source.

13: The microwave passive sensor of claim 1, wherein the microwave passive sensor provides Doppler information of a moving target based on reflected signals from the moving target and direct-path signals from a microwave source.

14: The microwave passive sensor of claim 13, wherein the moving target comprises a human, an animal, an object, a fluid, a human activity, a human gesture, a human vital sign or an animal vital sign.

15: The microwave passive sensor of claim 1, wherein the microwave passive sensor is tunable to different frequencies or scans a range of frequencies.

16: A method for passively detecting a movement of a target, the method comprising:
receiving signals using a microwave receiver;
amplifying the received signals using a low noise amplifier coupled to the microwave receiver;
separating the amplified received signals into a reflected signal from the target and a direct signal from a microwave source;
producing I/Q baseband signals from the reflected signal and the direct signal using a mixer;
amplifying the I/Q baseband signals using a baseband amplifier; and
detecting the movement of the target using the amplified I/Q baseband signals.

17: The method of claim 16, wherein the low noise amplifier comprises two cascaded low noise amplifiers.

18: The method of claim 16, wherein:
the mixer is radio frequency (RF) coupled to the low noise amplifier with a first capacitor; and
the baseband amplifier is RF coupled to the mixer with a second capacitor.

19: The method of claim 16, wherein the mixer comprises a diode coupled to ground.

20: The method of claim 16, wherein the mixer comprises a passive quadrature mixer.

21: The method of claim 16, wherein the mixer comprises:
a first resistor coupled between a voltage source and a mixer output;
an inductor coupled between the mixer output and a mixer input; and
a diode coupled between the mixer input and a ground.

22: The method of claim 16, wherein the baseband amplifier comprises:
a second resistor coupled between a voltage source and a negative input of an amplifier;
a third resistor and a fourth capacitor connected in parallel between the negative input of the amplifier and a ground;
a fourth resistor coupled between a mixer output and a positive input of the amplifier; and
a fifth resistor and a third capacitor connected in parallel between the positive input of the amplifier and an output of the amplifier.

23: The method of claim 16, wherein:
the amplified received signals are further amplified by a gain block amplifier coupled to the low noise amplifier; and
the amplified received signals are separated by a power divider coupled between the gain block amplifier and the mixer.

24. (canceled)

25: The method of claim 16, further comprising:
providing an analog to digital converter coupled to the baseband amplifier; and
providing a processor or computer coupled to the analog to digital converter.

26-27. (canceled)

28: The method of claim 16, further comprising providing Doppler information of a moving target based on reflected signals from the moving target and direct-path signals from a microwave source.

29. (canceled)

30: The method of claim 16, further comprising tuning the microwave passive sensor to different frequencies or scanning a range of frequencies

31: A system comprising:
a microwave passive sensor comprising a microwave receiver, a low noise amplifier coupled to the microwave receiver, a mixer coupled to the low noise amplifier, and a baseband amplifier coupled to the mixer;
a microwave source; and
a processor or computer coupled to the microwave passive sensor.

32-45. (canceled)
